(19) United States
(12) Patent Application Publication
(10) Pub. No.: US 2005/0092329 A1
(43) Pub. Date: May 5, 2005

(54) Nasal continuous positive airway pressure cannula device and securement for infants

(76) Inventor: Rosalinda C. Sta-Maria, North Brunswick, NJ (US)

Correspondence Address: Mr. Walter J. Tencza Jr., Suite 3, 10 Station Place, Metuchen, NJ, 08840, US

(21) Appl. No.: 10/696,664

(22) Filed: Oct. 29, 2003

(51) Int. Cl. A62B 7/00; A61M 15/08

(52) U.S. Cl. 128/207.18

(57) ABSTRACT

A nasal CPAP cannula device for infants designed for easy application and maintenance, using a securement method that requires only strips of adhesive tape and/or adhesive tape substitutes.

### FIELD OF THE INVENTION

This invention relates to improved methods and apparatus concerning devices for providing respiratory support.

### BACKGROUND OF THE INVENTION

CPAP, the acronym for continuous positive airway pressure, is a common modality of providing respiratory support to newborn infants with a variety of medical conditions such as respiratory distress syndrome, pneumonia, and chronic pulmonary insufficiency. By delivering gases such as air or oxygen-enriched air under a certain amount of pressure, CPAP keeps the infant's lungs in a slightly distended state thereby reducing the work of breathing; as well as helping the infant maintain oxygenation and eliminate carbon dioxide which is the waste product of respiration.

Several means of delivering CPAP to an infant have emerged in practice, each with advantages and disadvantages. One is via an endotracheal tube or a breathing tube introduced into the windpipe. While it is simple to maintain, it is invasive and predisposes the patient to respiratory infections. Another way is the nasopharyngeal tube which is passed through the nare until its tip is positioned in the posterior pharynx, as discussed in the article 37 Nasopahryngeal CPAP: A Nursing Art” in the September 2001 issue of The Journal of Neonatal Nursing. Although this method may be easy to secure, it can introduce microorganisms into an infant's upper airway and cause ulcerations of the nasal mucosa. A few other devices such as the face mask and the sealed box have fallen out of favor.

The most popular method of CPAP administration to infants is by means of a nasal cannula. Although many patents reveal securing methods for nasal cannula, they are not relevant to the present invention because such nasal cannulas are not intended for delivering pressurized gases, but are used for administering free-flowing gases while attached to a light, slim tubing coming from a gas source. Being light in weight, the methods of securing them do not present the same degree of difficulty as nasal cannula used for delivering continuous positive airway pressure or pressurized gas. Generally CPAP cannulas of the prior art are bulky and require connection to heavy, large-bore circuit tubings from a pressure-generating machine; such type of tubings being necessary for humidifying and heating the pressurized gas it must carry, because unheated and unhumidified gases are corrosive to the tissues of the airway and the lungs. The circuit may have other attachments; usually, a pressure sensor and a temperature sensor for monitoring the pressure and the temperature of gases being delivered to the infant. These attachments add to the weight of the circuit tubings.

Practitioners in the field often refer to CPAP cannula described in the preceding paragraph as “nasal prongs”, differentiating it from the slim nasal cannula which simply attaches to a thin oxygen tubing coming from an oxygen source through a flow meter gauge. U.S. Pat. No. 5,271,391 to Graves discloses a slim, light weight nasal cannula for CPAP which is attached to a thin tubing and can be secured with pieces of adhesive on the cheeks of the infant. In U.S. Pat. No. 5,271,391, the narrow tubing does not allow for adequate humidification and heating of the pressurized gas being delivered to the patient, and the sheer length of the tubing can add substantially to the dead space in the system which can hamper gas exchange. Therefore, that system has not been a common choice for CPAP administration.

CPAP prongs are the most common device for this purpose and have been applied on infants using hats, straps with hook-and-loop fasteners, and foam blocks as disclosed in Ackerman, et al. U.S. Pat. No. 4,774,946. Using a skull cap with ties is disclosed in Dali U.S. Pat. No. 4,367,735. A head cradle assembly with head-restraining plates is disclosed in Ko, et al. U.S. Pat. No. 4,249,527, and a premature baby headband is disclosed in Tumolo U.S. Pat. No. 5,188,101. A nasal CPAP assembly made in New Zealand by Fisher & Paykel Healthcare uses a hat, a strap with hook-and-loop fasteners, ties, and foam blocks. Winthrop, et al. U.S. Pat. No. 5,682,881 teaches a “foam securement strip which adhesively attaches to the upper lip of the individual,” still on a 600-gram premature infant, even a thin foam layer can occlude the infant's nares and not leave enough room for the insertion of the prongs.

Of the aforementioned CPAP devices available in the market, none have an effective method of securement. A head cradle assembly with head-restraining plates is cumbersome and has not been around in recent years. Hats, headbands, and straps easily come off the round head, even more so when the infant is active and squirming. Tubings slip through foam blocks, and ties do not serve their purpose once the hat, straps, or headband to which they are attached are out of place. It is not unusual to find the prongs over the eyes, the mouth, or anywhere else, except where they should be which is the infant's nostrils. In those instances that they are in place, the prongs are pushed up violently against the infant's nose giving the infant a pug-nose appearance and a deformed nasal septum. The frustration of almost continually re-applying it to the infant's nares and the failure to keep them there have become the bane of a practitioner's work and an on-going challenge to his or her clinical skills, not to mention the frequent interruptions to the infant's rest and sleep. There is also the concern of the infant not getting the respiratory support he or she needs which can lead to collapsed lungs and to low oxygen levels in his or her blood, among other things. Other distressing complications of the prior art devices include deformed skull from headbands, dents on the temples from foam blocks, deformed nasal septum, and irritated nares. These complications arise from the inadequacy of the securing methods of prior art devices to maintain nasal CPAP prongs in place and in neutral position, meaning the prongs not impinging or exerting undue pressure on the surrounding tissues of the face.

### SUMMARY OF THE INVENTION

The present invention discloses a nasal CPAP cannula for infants that is designed and configured to be secured by means of strips of adhesive tape and/or adhesive tape substitutes. Furthermore, its securement method does not require the use of hats, headbands, straps with hook-and-loop fasteners, or ties. One embodiment of the present invention is comprised of a nose piece and a head piece.

It is the object of this invention to provide a nasal CPAP cannula device that is simple for the practitioner to use, secure, and maintain. Another object of this invention is to minimize the complications to infants that are resultant of the difficulty in securing nasal CPAP cannulas of prior arts.

The present invention, in one or more embodiments, is comprised of a nose piece and a head piece. The nose piece may be comprised of a first and second hollow tubes, which protrude out from the nose piece on a first side of the nose piece. First ends of the first and second hollow tubes can be inserted into first and second nostrils, respectively, of an individual. The nose piece may be hollow, cylindroid, and be comprised of a flexible material. The nose piece may also be comprised of third and fourth hollow tubes which protrude out from the nose piece on a second side of the nose piece. Air can flow through the first hollow tube into a cavity of a body portion of the nose piece and then into the third hollow tube. Air can also flow through the second hollow tube into the cavity and then into the fourth hollow tube.

The head piece may be comprised of two or more tubes and an attachment device for attaching the head piece at or near a forehead of the individual. The head piece, may for example be comprised of fifth, sixth, seventh, eighth, ninth, and tenth tubes. The tenth tube may be connected to the eighth tube, which may be connected to the sixth tube, which may be connected to the fourth tube of the nose piece. The ninth tube may be connected to the seventh tube, which may be connected to the fifth tube, which may be connected to the third tube of the nose piece. The ninth and tenth tubes may be connected to a ventilator circuit, which may supply air to the nostrils of the individual.

The nose piece may include an attachment device, such as a flap portion, which may be attached at or near the upper lip of an individual. The attachment device may be attached by an adhesive.

The first and second hollow tubes of the nose piece may have a first diameter and the third and fourth hollow tubes of the nose piece may have a second diameter, wherein the first diameter is substantially smaller than the second diameter.

The present invention may also include attaching a nose piece to an individual at or near the upper lip of the individual and attaching a head piece to the individual at or near the forehead of the individual.

### BRIEF DESCRIPTION OF THE DRAWINGS

FIG. 1A shows a perspective view of a nose piece in accordance with an embodiment of the present invention;

FIG. 1B shows a perspective view of various tubes used for the nose piece of FIG. 1A;

FIG. 2A shows a top view of a tube for use with an embodiment of the present invention with the tube in an expanded state;

FIG. 2B shows a right side view of the tube of FIG. 2A;

FIG. 2C shows a left side view of the tube of FIG. 2A;

FIG. 2D shows a top view of the tube of FIG. 2A with the tube in a compressed state;

FIG. 3A shows a top view of another tube for use with an embodiment of the present invention;

FIG. 3B shows a right side view of the tube of FIG. 3A;

FIG. 4A shows a top view of four tubes each of which is the same as the tube in FIG. 2A and two tubes each of which is the same as the tube in FIG. 3A; with two of the tubes corresponding to the tube in FIG. 3A connected to one of the tubes corresponding to the tube in FIG. 2A; and the other two tubes corresponding to the FIG. 3A tube connected to the other tube corresponding to the tube in FIG. 2A

FIG. 4B shows a top view of an apparatus or head piece comprised of the six tubes of FIG. 4A connected together by a device;

FIG. 4C shows a bottom view of the apparatus of FIG. 4B;

FIG. 5 shows a side view of an assembled nose piece and head piece applied on the face of an infant, in position for use, along with a ventilator circuit referred to in block diagram format; and

FIG. 6 shows a top view of the assembled nose piece and head piece applied to the face of the infant, in position for use, along with the ventilator circuit referred to in block diagram format.

### DETAILED DESCRIPTION OF THE DRAWINGS

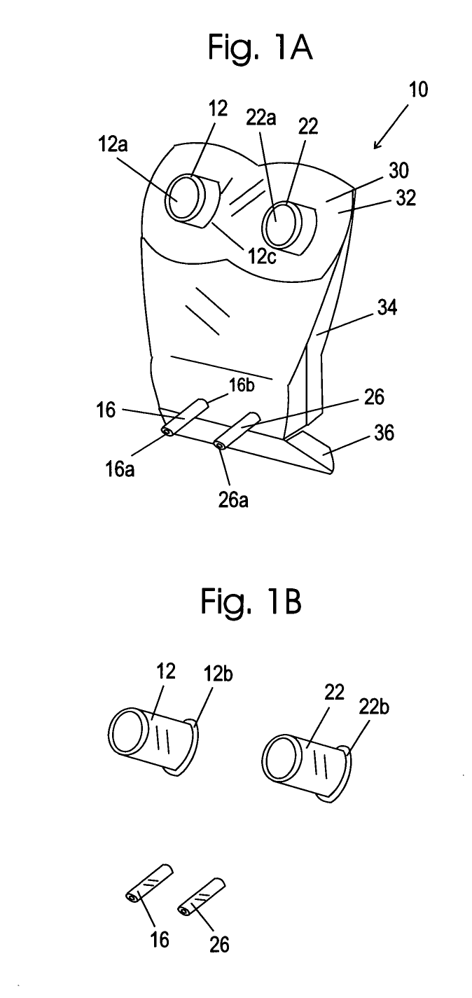

FIG. 1A shows a perspective view of a nose piece 10 in accordance with an embodiment of the present invention. FIG. 1B shows a perspective view of various tubes used for the nose piece 10 of FIG. 1A. The nose piece 10 includes hollow tubes 12, 16, 22, and 26. The nose piece 10 also includes a material or device 30, which is comprised of portions 32, 34, and 36. The hollow tubes 12 and 22 are held by the portion 32 of the material or device 30. The hollow tubes 16 and 26 are held by the portion 34 of the material or device 30. The hollow tubes 16 and 26 may be called prongs. The device 30 is hollow such that air passing into opening 12a and opening 22a goes into and through tubes 12 and 22, respectively, and into and through a cavity in device 30 The air from the cavity within the device 30 comes out of either tube 16 or 26 through openings 16a or 26a.

Except for the two superior projections or hollow tubes 12 and 22 which are typically made of hard plastic and bonded or dip molded in the main body of the material or device 30, the rest of the nose piece 10 is typically constructed of light, pliable, synthetic polymer such as polyvinyl chloride (PVC) and silicone rubber, and made by a molding process commonly used in the manufacture of many plastic items.

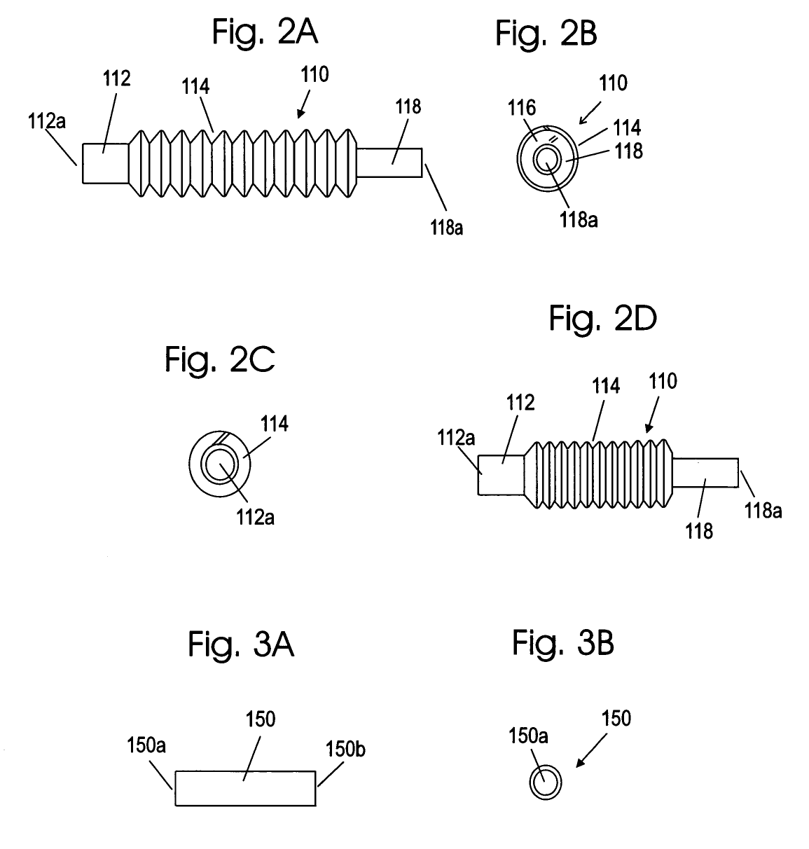

FIG. 2A shows a top view of a hollow tube 110 for use with an embodiment of the present invention with the tube 110 shown in an expanded state. FIG. 2B shows a right side view of the tube 110 of FIG. 2A. FIG. 2C shows a left side view of the tube 110 of FIG. 2A. FIG. 2D shows a top view of the tube 110 of FIG. 2A with the tube 110 in a compressed state. The tube 110 has an opening 112a at one end and an opening 118a at its other end. The tube 110 has a portion 112, which typically has a slightly greater diameter than a portion 118. The tube 110 has a portion 114, which is flexible and which can be placed in compressed state as in FIG. 2D or in an expanded state as in FIG. 2A.

FIG. 3A shows a top view of a hollow tube 150 for use with an embodiment of the present invention. FIG. 3B shows a right side view of the tube 150 of FIG. 3A. The tube 150 has an opening 150a at one end and an opening 150b at its other end.

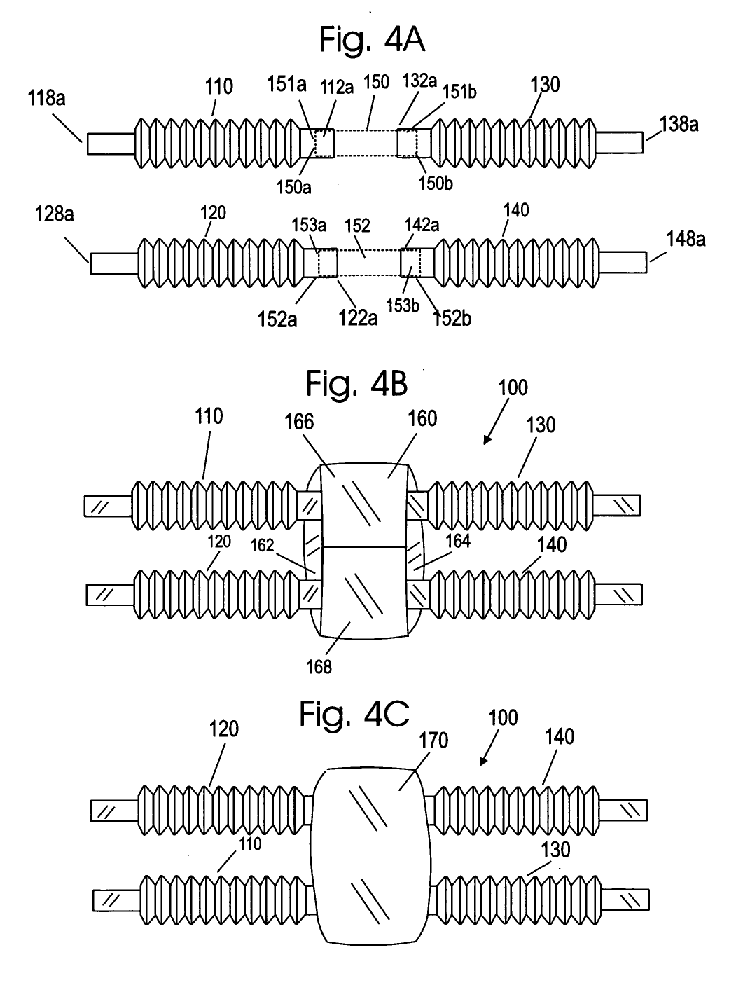

FIG. 4A shows a top view of hollow tubes 110, 120, 130, and 140 each of which is the same as tube 110 shown in FIGS. 2A-2D. Also shown, by dashed lines are tubes 150 and 152, each of which is the same as tube 150 shown in FIGS. 3A-B. In FIG. 4A, end 151a of tube 150 is inserted into opening 112a of the tube 110 and end 151b of the tube 150 is inserted into an opening 132a of the tube 130. The tubes 110, 150 and 130 can be fixed together in this configuration by friction, glue, taping, or in any other manner. Similarly, end 153a of tube 152 is inserted into the opening 122a of tube 120 and end 153b of the tube 152 is inserted into the opening 152b of the tube 140.

FIG. 4B shows a top view an apparatus or head piece 100 comprised of tubes 110, 120, 130, 140, 150, and 152 connected together by a material or device 160. The material or device 160 may be comprised of portions 162, 164, 166, 168 and 170. The material or device 160 is typically constructed of light, pliable, synthetic polymer such as PVC (polyvinylchloride) and silicone rubber. FIG. 4C shows a bottom view of the apparatus or head piece 100 of FIG. 4B. The material or device 160 covers the tubes 150 and 152.

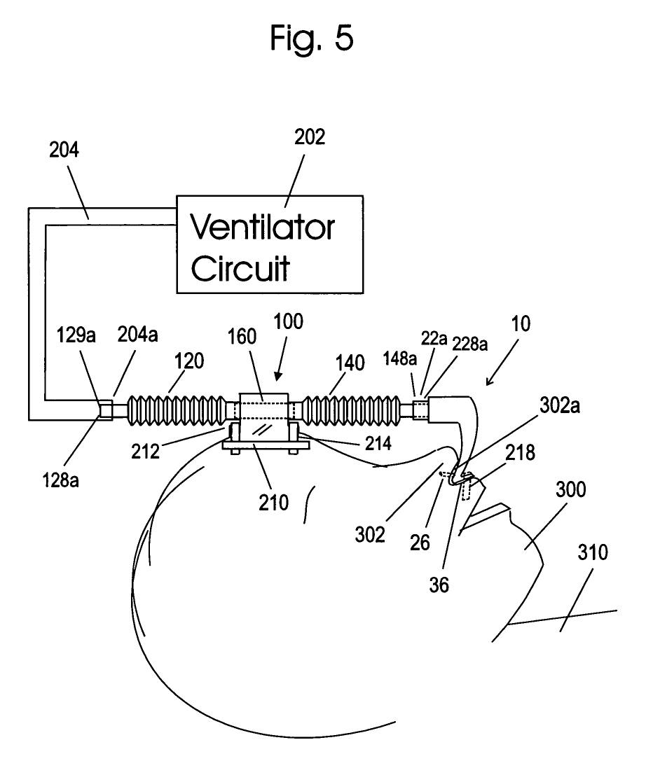

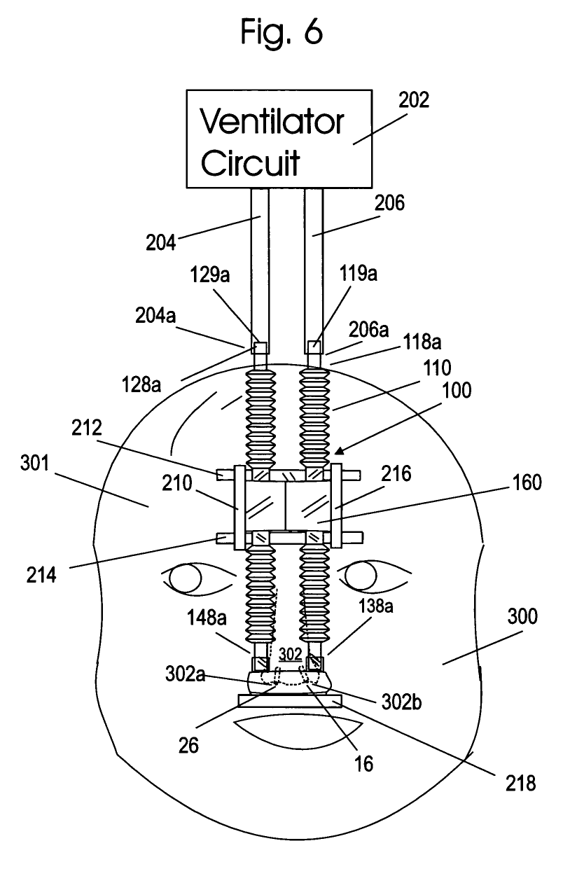

FIG. 5 shows a side view of an assembled nose piece 10 and head piece 100 applied on the face of an infant 300, in position for use, along with a ventilator circuit 202 referred to in block diagram format. FIG. 6 shows a top view of the assembled nose piece 10 and head piece 100 applied to the face of the infant 300 in position for use, along with the ventilator circuit 202 referred to in block diagram format. Referring to FIGS. 5 and 6, the ventilator circuit is connected by tubes 204 and 206 to end 129a of tube 120 and end 119a of tube 110. The ends 129a and 119a are shown inserted into ends 204a and 206a of tubes 204 and 206, respectively.

The tubes 16 and 26 of the nose piece 10 are shown by dashed lines as being inserted into the nostrils 302a and 302b of the nose 302 of the infant 300. Ends 148a and 138a of the tubes 140 and 130 are shown inserted into the openings 12a and 22a, respectively, of the nose piece 10.

The device 160 is shown taped by adhesive strips 210, 212, 214, and 216 to the forehead 301 of the infant 300. The portion or flap 36 of the nose piece 10 is shown taped by adhesive strip 218 above the upper lip 303 of the infant 300. The strip of adhesive 218 is long enough to secure the nose piece 4 in position in front of the nasal openings 302a and 302b, extending beyond extension or portion 36 of the nose piece 10 onto the skin of the upper lip 303.

In operation, air, oxygen-enriched air, or other therapeutic gas is pumped by ventilator circuit 202 of FIGS. 5 and 6 into hollow tubes 204 and 206. The air, for example, from tube 204 flows into tube 120, then into tube 152, then into tube 140, then into tube 22 of the nose piece 10. The air then flows into a cavity inside the hollow body of device 30 of the nose piece 10. The air from the cavity may then flow into then tube 26 or tube 16. If the air flows into tube 26, it will flow into the nostril 302a. Similarly the air from tube 206 flows into tube 110, then into tube 150, then into tube 130, then into tube 12 of the nose piece 10. The air then flows into the cavity inside the hollow body of device 30 of the nose piece 10. The air from the cavity may flow into either tube 16 or 26. Air flowing into tube 16, flows into nostril 302b.

With the exception of the nasal cannula assembly made by Fisher & Paykel, nasal CPAP cannulas of the prior art were designed for the main body to lie horizontally across the face in the area below the nose so its prongs are inside the patient's nostrils . An embodiment of the current invention, as shown by FIGS. 5 and 6, discloses a main body of a nose piece 10 designed for positioning vertically in relation to the horizontal plane 310 (shown in FIG. 5) of the infant's body lying on the infant's back. The embodiment of the present invention further discloses an H-shaped head piece or apparatus 100, shown in FIGS. 4B and 4C, which is adhesively mounted on the forehead 301 of the infant 300 and adapted to communicate with the nose piece 10 on one side, and with the ventilator circuit 202 on the other side.

The head piece or apparatus 100 functions as a staging mount and a coupler between the nose piece 10 and the ventilator 202 or other pressure-generating machine or machines. The device or material 160 of the head piece 100 may be made of the same light, pliable, synthetic polymer as the nose piece 10, having an incurvature on its underside surface or portion 170 so as to conform to the contour of the forehead 301 of the infant 300. In the platform or device or material 160 are embedded the two rigid hollow tubes 150 and 152, respectively serving as a conduits between elongations or tubes 110 and 130 and 120 and 140. These four elongations or tubes 110, 120, 130, and 140, are typically made of corrugated, bendable, compressible, and extendable hollow tubes which are readily available and currently in use and which can be manipulated to adjust angle and length for the purpose of releasing tension on the nose piece 10 and the headpiece 100 thereby promoting securability. In at least one embodiment, the elongations or tubes 110, 120, 130, and 140 connecting to the ventilator circuit 202 should be long enough to go over the head of the infant 300 so that their weight will not bear on the head of the infant 300. In at least one embodiment of the present invention the elongations or tubes 110, 120, 130, and 140 connecting to the nosepiece 10 should be short enough to fit a 500-gram infant at its fully compressed state, i.e. when the tubes 110, 120, 130, and 140 are fully compressed. For a big infant who requires a longer connection between the nose piece 10 and the head piece or apparatus 100 in spite of the elongations being fully extended, standard plastic adaptors may be used to add length.

Around platform material or device 160 are thin extensions or portions 162, 164, 166, and 168 shown in FIG. 4B over which adhesive strips 212, 214, 216, and 218, respectively, are attached to secure the headpiece or apparatus 100 onto the forehead 301 of the infant 300. The portions or extensions 162, 164, 166, and 168 may take a variety of shapes and number; however, the device 160 may have the shape of a four-leaf clover. Alternatively, the extensions may be continuous around the platform or device 160.

The head piece 100 in the present embodiment is illustrated as one integral unit for ease of use; alternatively, the elongations or tubes 110, 120, 130, and 140 of the head piece 100 may be supplied as separate parts, adapted to be assembled with the platform or device 160 at the point and time of use, by connecting them to the rigid tubes 150 and 152. In at least one embodiment, projections or tubes 12 and 22 of the nose piece 10 may be female adaptors and will connect to male adaptors such as 148a and 138a of elongations or tubes 130 and 140. Also, female adaptors or tubes 110 and 120 may connect with male adaptors of tubes 204 and 206 and then to the ventilator circuit 202. The tubes 204 and 206 may be comprised of standard pediatric corrugated tubings.

Additionally, the projections or tubes 16 and 26 in the lower portion of the nose piece 10 will be manufactured in graduated sizes to accommodate the smallest premature infant to the biggest term infant.

In view of the sensitive skin of many infants, in particular, the fragile skin of premature infants, the following suggestions are presented for the choice and use of adhesive strips for strips of adhesive 210, 212, 214, 216, and 218 and/or adhesive substitute strips in the implementation of the securing method of the presently disclosed nasal CPAP cannula device:
1. Consider using hydrocolloid-based adhesive such as NeoBond™ made by Neotech Products, Inc. which claims to adhere to the Skin Care Guidelines put forth by the National Association of Neonatal Nurses (NANN) for the strips 210, 212, 214, 216, and 218 and with further claims that this product provides long term attachment but prevents epidermal stripping
2. Consider exploring other adhesive substitutes such as those used in securing ostomy appliances such as the Hollister Skin Barriers
3. If regular adhesive strips are to be used, consider applying first a protective layer of adhesive on the infant's skin such as a clear breathable occlusive dressing made by Johnson&Johnson Medical, or of pink tape such as the latex-free Hy•Tape® made by Hy•Tape International that claims a property of this particular adhesive tape to adhere to wet, oily, hairy skin and to be removed painlessly. It further claims to its being waterproof and washable, and, therefore,its being perfect for extended wear.

Other nursing concerns as relate to the care of an infant using nasal CPAP cannula of the present invention are addressed as follows:
1. To remove the nose piece 10 momentarily for suctioning, disconnect nose piece projections or tubes 12 and 22 from the head piece elongation adaptors 148a and 138a; then tilt the nose piece 10 backwards. Alternatively, the adhesive strip 218 over nose piece 10 tab-like extension or portion 36 can be removed so that the nose piece 10 can be lifted away from the nasal openings 302a and 302b. After the procedure, a new adhesive strip can be applied to re-secure the nose piece 10 in position.
2. Since the tension on the elongations or tubes 110, 120, 130, and 140 can compromise the secure placement of both the nose piece 10 and the head piece 100, the elongations or tubes 110, 120, 130, and 140 on both sides of the head piece 100 should be manipulated and adjusted to remove such tensions.
3. For a very small infant with a short upper lip to forehead distance, the headpiece or apparatus 100 may need to be positioned farther back on the forehead 301, and the use of adhesive strips on that side of the head piece 100 may be omitted if the head piece extension reaches beyond the infant's hairline. This is to avoid taping on the infant's hair.
4. A hat may be used for temperature maintenance by making a slit in front that is big enough and long enough for the elongations 110 and 120 to exit through.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

1. An apparatus comprising
a nose piece comprising
a body portion;
a first hollow tube protruding out from the body portion; and
a second hollow tube protruding out from the body portion;
a first device for attaching the first and second hollow tubes to an individual's head;
wherein a first end of the first hollow tube can be inserted into a first nostril of an individual;
wherein a first end of the second hollow tube can be inserted into a second nostril of the individual;
wherein air can flow through the first hollow tube into the first nostril and through the second hollow tube into the second nostril; and
wherein the body portion is flexible.

2. The apparatus of claim 1 wherein
the body portion is comprised of a pliable synthetic polymer.

3. The apparatus of claim 1 wherein
the body portion is hollow.

4. The apparatus of claim 2 wherein
the pliable synthetic polymer is comprised of polyvinyl chloride and silicone rubber.

5. The apparatus of claim 1 wherein
the body portion is cylindroid.

6. An apparatus comprising
a nose piece comprising
a body portion;
a first hollow tube protruding out from the body portion; and
a second hollow tube protruding out from the body portion;
a first device for attaching the first and second hollow tubes to an indivilual's head;
wherein a first end of the first hollow tube can be inserted into a first nostril of an individual;
wherein a first end of the second hollow tube can be inserted into a second nostril of the individual;
wherein air can flow through the first hollow tube into the first nostril and through the second hollow tube into the second nostril; and
wherein
the nose piece is further comprised of a flap portion.

7. The apparatus of claim 6 further comprising
an adhesive strip for taping the flap portion of the nose piece to the individual.

8. The apparatus of claim 1 further comprising
a third hollow tube;
a fourth hollow tube; and
wherein the third and fourth hollow tubes protrude out from the body portion of the nose piece.

9. The apparatus of claim 8 wherein
the first and second hollow tubes have a first diameter; and
the third and fourth hollow tubes have a second diameter; and
wherein the first diameter is substantially smaller than the second diameter.

10. The apparatus of claim 6 wherein
the flap portion can be attached to skin at or near an upper lip of the individual while the first end of the first hollow tube is in the first nostril and the first end of the second hollow tube is in the second nostril, of the individual.

11. The apparatus of claim 9 further comprising
a fifth hollow tube and a sixth hollow tube; and
wherein the fifth and sixth hollow tubes are connected to the third and fourth hollow tubes, respectively; and further comprising
a seventh hollow tube and an eighth hollow tube;
wherein the seventh and the eighth hollow tubes are connected to the fifth and the sixth hollow tubes, respectively, and the seventh and the eighth hollow tubes are attached to a second device for attaching the apparatus to the individual's head;
and wherein the first device is adapted to attach the apparatus at or near the upper lip of the individual and the second device is adapted to attach the apparatus at or near the forehead of the individual.

12. The apparatus of claim 11 further comprising
ninth and tenth hollow tubes;
wherein the ninth and tenth hollow tubes are connected to the seventh and eighth hollow tubes, respectively.

13. The apparatus of claim 1 further comprising
a ventilator circuit for supplying air to and through the first and second hollow tubes, and to the first and second nostrils.

14. The apparatus of claim 12 further comprising
a ventilator circuit for supplying air to and through the ninth and tenth hollow tubes and to the first and second nostrils.

15. The apparatus of claim 11 further comprising
a first adhesive for attaching the first device at or near the upper lip of the individual; and
a second adhesive for attaching the second device at or near the forehead of the individual.

16. A method comprising the steps of
attaching first and second hollow tubes to an individual's head;
inserting a first end of the first hollow tube into a first nostril of an individual;
and inserting a first end of the second hollow tube into a second nostril of the individual;
and wherein the first and second hollow tubes protrude out from a body portion of a nose piece and wherein the first and second hollow tubes are attached to the individual's head through the nose piece, which is attached at or near the upper lip of the individual; and
wherein the body portion of the nasal piece is flexible.

17. The method of claim 16 further comprising
connecting a third hollow tube to the first hollow tube; and
connecting a fourth hollow tube to the second hollow tube;
attaching the third hollow tube at or near the forehead of an individual;
and attaching the fourth hollow tube at or near the forehead of an individual.

18. The method of claim 16 wherein
the body portion is comprised of pliable synthetic polymer.

19. A method comprising the steps of
attaching first and second hollow tubes to an individual's head;
inserting a first end of the first hollow tube into a first nostril of an individual;
and inserting a first end of the second hollow tube into a second nostril of the individual;
and wherein the first and second hollow tube protrude out from a body portion of a nose piece and wherein the first and second hollow tubed are attached to the individual's head through the nose piece, which is attached at or near the upper lip of the individual; and
wherein
the nose piece includes a flap portion.

20. The method of claim 19 wherein
the nose piece is attached by an adhesive, which adheres the flap portion of the nose piece to the individual at or near an upper lip of the individual.

21. The method of claim 16 further comprising
attaching third and fourth hollow tubes to the body portion of the nose piece; and
wherein the third and the fourth hollow tubes protrude out from the body portion of the nose piece.

22. The method of claim 21 wherein
the first and second hollow tubes have a first diameter; and
the third and fourth hollow tubes have a second diameter; and
wherein the first diameter is substantially smaller than the second diameter.

23. The method of claim 22 further comprising
attaching a fifth hollow tube to the third hollow tube; and
attaching a sixth hollow tube to the fourth hollow tube.

24. The method of claim 23 further comprising
supplying air to the fifth hollow tube and thereby to the third, and first hollow tubes and to the first nostril;
and supplying air to the sixth hollow tube and thereby to the fourth, and second hollow tubes and to the second nostril.

25. The method of claim 24 further comprising
attaching a seventh hollow tube to the fifth hollow tube;
attaching an eighth hollow tube to the sixth hollow tube; and
attaching the seventh hollow tube and eighth hollow tubes at or near the individual's forehead.

26. The method of claim 25 further comprising
attaching ninth and tenth hollow tubes to the seventh and eighth hollow tubes, respectively.

27. The method of claim 16 further comprising
supplying air to and through the first and second hollow tubes, and to the first and second nostrils.

28. The method of claim 26 further comprising
supplying air to and through the ninth and tenth hollow tubes, and to the first and second nostrils.

29. The method of claim 25 wherein
the seventh and eighth hollow tubes are attached at or near the individual's forehead by an adhesive.

30. The apparatus of claim 6 wherein
the flap portion is flexible.

31. The apparatus of claim 30 wherein
the flap portion is comprised of a pliable synthetic polymer.

32. The apparatus of claim 31 wherein
the pliable synthetic polymer is comprised of polyvinyl chloride and silicone rubber.

33. An apparatus for use In supplying air to an individual comprising
a first device;
a second device;
a means for attaching the first device to an individual's forehead;
a means for attaching the second device to an area at or near the individual's upper lip;
first and second hollow tubes connected to the second device, wherein the first and second hollow tubes have first and second ends, respectively, which can be inserted into first and second nostrils, respectively, of an individual;
wherein the first device is connected to the second device so that air can flow from the first device to the second device and to the first and second hollow tubes;
wherein the means for attaching the first device does not circle a head of the individual in order to attach the first device; and
wherein the means for attaching the second device does not circle the head of the individual in order to attach the second device.

34. The apparatus of claim 33 wherein
the means for attaching the first device is located substantially only at or near the individual's forehead; and
and the means for attaching the second device is located substantially only at or near the individual's upper lip.
